(19) United States
(12) Patent Application Publication
(10) Pub. No.: US 2019/0153427 A1
(43) Pub. Date: May 23, 2019

(54) DETERMINATION OF RNA IN BLOOD OR OTHER FLUIDS

(71) Applicant: President and Fellows of Harvard College, Cambridge, MA (US)

(72) Inventors: David A. Weitz, Bolton, MA (US); Huidan Zhang, Cambridge, MA (US); Nai Wen Cui, Cambridge, MA (US)

(73) Assignee: President and Fellows of Harvard College, Cambridge, MA (US)

(21) Appl. No.: 16/315,194

(22) PCT Filed: Jul. 7, 2017
(86) PCT No.: PCT/US2017/041051

§ 371 Date: Jan. 4, 2019

Related U.S. Application Data
(60) Provisional application No. 62/360,076, filed on Jul. 8, 2016.

(51) Int. Cl. C12N 15/10; C01C 1/24; C01C 1/14; C12N 9/22

(52) U.S. Cl. CPC C12N 15/101; C12N 9/22; C01C 1/14; C01C 1/24

(57) ABSTRACT

The present invention generally relates to systems and methods for determining RNA in blood or other fluids. In certain embodiments, blood or other fluids may be treated to isolate or separate RNA, for example, from DNA, cells, and other material. In some cases, the RNA may arise from bacteria or other pathogens or foreign organisms that may be found within the blood or other fluid. In some cases, RNA stabilizing reagents, such as ammonium sulfate, may be added to stabilize RNA, then cells within the blood may be lysed to release the RNA (and other materials) from the cells, thereby producing a lysate. The lysate may be treated, e.g., to separate nucleic acids from other components within the lysate, and in some cases, DNA may be degraded, e.g., using DNAses or other suitable enzymes, leaving behind the RNA. The RNA can then be studied, purified, analyzed, amplified, stored, or the like.

### RELATED APPLICATIONS

This application claims the benefit of U.S. Provisional Patent Application Ser. No. 62/360,076, filed Jul. 8, 2016, entitled “Determination of RNA in Blood or Other Fluids,” by Weitz, et al., incorporated herein by reference in its entirety.

### GOVERNMENT FUNDING

This invention was made with government support under Grant Nos. DMR-1420570 and DMR-1310266 awarded by the National Science Foundation. The government has certain rights in the invention.

### FIELD

The present invention generally relates to systems and methods for determining RNA in blood or other fluids.

### BACKGROUND

The vast majority of bacteria do not cause disease, although many bacteria are actually helpful and even necessary to good health. Millions of bacteria normally live on the skin, in the intestines, and elsewhere within the body. However, some bacterial diseases can result when the harmful bacteria get into an area of the body that is normally sterile, such as the bladder, or when they crowd out helpful bacteria in places such as the intestines. Harmful or pathogenic bacteria include, for example, Neisseria meningitidis, which can cause meningitis, Streptococcus pneumoniae, which can cause pneumonia, and Staphylococcus aureus, which can cause a variety of infections. Other examples of pathogenic bacteria include Helicobacter pylori, which can cause gastric ulcers, and Escherichia coli or Salmonella, which can both cause food poisoning.

Traditional methods for detection of bacteria in blood and other fluids are often not sensitive or fast enough to achieve early diagnosis, which can prevent certain infectious diseases from being effectively treated. Accordingly, improvements are needed.

### SUMMARY

The present invention generally relates to systems and methods for determining RNA in blood or other fluids. The subject matter of the present invention involves, in some cases, interrelated products, alternative solutions to a particular problem, and/or a plurality of different uses of one or more systems and/or articles.

In one aspect, the present invention is generally directed to a method comprising adding an RNA-stabilizing reagent to a blood sample, lysing cells within the blood sample to produce a lysate, degrading DNA within the lysate, and separating RNA from the lysate.

In another aspect, the present invention is generally directed to lysing red blood cells in a blood sample, adding an RNA-stabilizing reagent to the blood sample, lysing cells within the blood sample to produce a lysate, degrading DNA within the lysate, adding the lysate to a column containing silica, removing non-nucleic acid species from the column, and thereafter, eluting RNA from the column.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic showing extraction of RNA, in accordance with one embodiment of the invention; and

FIG. 2 illustrates detection of RNA, in another embodiment of the invention.

### DETAILED DESCRIPTION

The present invention generally relates to systems and methods for determining RNA in blood or other fluids. In certain embodiments, blood or other fluids may be treated to isolate or separate RNA, for example, from DNA, cells, and other material. In some cases, the RNA may arise from bacteria or other pathogens or foreign organisms that may be found within the blood or other fluid. In some cases, RNA stabilizing reagents, such as ammonium sulfate, may be added to stabilize RNA, then cells within the blood may be lysed to release the RNA (and other materials) from the cells, thereby producing a lysate. The lysate may be treated, e.g., to separate nucleic acids from other components within the lysate, and in some cases, DNA may be degraded, e.g., using DNAses or other suitable enzymes, leaving behind the RNA. The RNA can then be studied, purified, analyzed, amplified, stored, or the like.

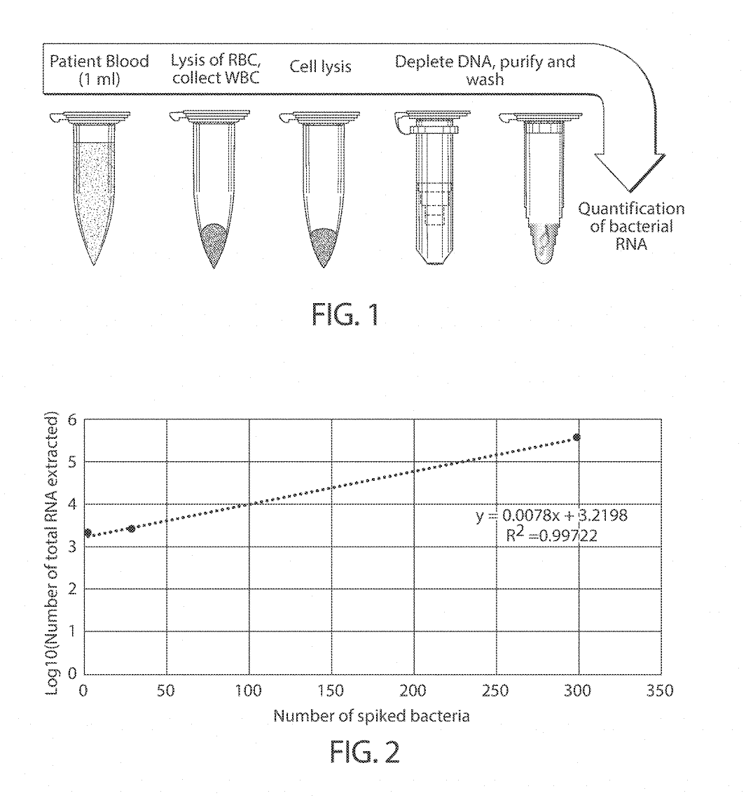

One non-limiting example of an RNA isolation protocol is illustrated in FIG. 1. In this example, a sample to be determined, such as patient blood, is collected. As discussed below, this is by way of example, and other fluids may be determined in other embodiments. In this example, cells (e.g., white blood cells, pathogens such bacteria, etc.), are collected via centrifugation, optionally treated with one or more RNA-stabilizing reagents, and lysed to release their RNA. The DNA released from the lysed cells may be degraded in some fashion, e.g., using a reaction that selectively degrades DNA relative to RNA. For instance, in some cases, the RNA-stabilizing reagents may prevent or decrease the likelihood of the RNA from being degraded, relative to DNA, or various enzymes that preferentially act on DNA relative to RNA may be used, e.g., a DNAse or a restriction endonuclease. After such depletion of DNA, the RNA may then be studied in some fashion. For example, the RNA may be sequenced or compared against RNA known to arise from bacteria to determine the presence and/or concentration of such bacteria within the sample. In some cases, RNA not arising from a known bacteria may be determined, e.g., to determine new species of bacteria or other species (e.g., a pathogenic species). As yet another non-limiting example, the RNA may be isolated and/or sequenced to determine the activity of cells within the sample, e.g., the transcriptome of various cells within the sample.

The fluid sample may be any suitable fluid. Examples of fluid include, but are not limited to, cell culture fluid, water, saline, soil samples or other environmental samples, blood, or another bodily fluid, such as perspiration, saliva, plasma, tears, lymph, urine, plasma, or the like. In some cases, the fluid is an artificial fluid, e.g., cell culture fluid. In some cases, the fluid may arise, from a human or any other organism, e.g., a non-human mammal. In some cases, a sample of tissue, such as biopsy, may be taken and then homogenized or processed to separate cells, which may be used to form a suitable fluid, for instance, through admixture with saline. The fluid, in some embodiments, may be a relatively complex or biological mixture, e.g., containing a variety of cells and/or species, and in some cases, is not well-defined, e.g., unlike saline or a simple cell culture.

In some cases, one or more RNA-stabilizing reagents may be added to the fluid to stabilize the RNA therein. The RNA-stabilizing reagent may be added to intact cells, or the cells within the fluid may be lysed prior to adding the RNA-stabilizing reagent. In some cases, the RNA-stabilizing reagent may be able to enter intact cells.

In one set of embodiments, the RNA-stabilizing reagent may include ammonium sulfate. The ammonium sulfate may be added, for example, such that the final concentration of ammonium sulfate in the fluid is no more than about 100 g/100 ml, no more than about 50 g/100 ml, no more than about 20 g/100 ml, no more than about 10 g/100 ml, or no more than about 5 g/100 ml. In some cases, the final concentration of ammonium sulfate may be no more than about 64 mM, no more than about 50 mM, no more than about 32 mM, no more than about 10 mM, no more than about 5 mM, etc. In addition, several RNA-stabilizing reagents may be obtained commercially, including RNAprotect Cell Reagent (Qiagen) or RNAlater (Thermo Fisher). Other examples of RNA-stabilizing reagents include those discussed in U.S. Pat. Appl. Pub. No. 2002/0115851.

In some embodiments, cells within the fluid may be lysed, for example, to release RNA and other contents from the cell, thereby producing a cell lysate. A variety of techniques can be used to lyse cells, such as exposure to a lysing chemical or a cell lysis reagent (e.g., a surfactant such as Triton-X or SDS, an enzyme such as lysozyme, lysostaphin, zymolase, cellulase, mutanolysin, glycanases, proteases, mannase, proteinase K, etc.), or a physical condition (e.g., ultrasound, ultraviolet light, mechanical agitation, etc.). Still other examples include chaotropic salts, detergents or alkaline denaturation.

For example, in one set of embodiments, cells may be lysed by exposure to compounds such as enzymes (e.g., proteinases such as Proteinase K), lysozymes, EDTA (ethylenediaminetetraacetatic acid), surfactants (e.g., Tris-HCl), lysis buffers (e.g., Buffer RLT from Qiagen), guanidine isothiocyanates, beta-mercaptoethanol, or the like. Many such compounds can be readily obtained commercially. As another example, red blood cells may be lysed by exposing the red blood cells to compounds such as ammonium chloride, EDTA (ethylenediaminetetraacetatic acid), sodium bicarbonate, or the like. In addition, it should be understood that more than one method may be used to lyse cells. For example, a sample of blood may be exposed to an erythrocyte lysis buffer, one or more enzymes, and/or one or more mechanical techniques in order to lyse cells within the blood. If more than one technique is used, they may occur in any suitable order, and before, during, and/or after other techniques discussed herein, e.g., adding an RNA-stabilizing reagent.

According to one set of embodiments, DNA within the lysate may be degraded. In some cases, the DNA may be degraded using techniques that are selective for DNA, relative to RNA, thereby making it easier to determine the RNA. In some cases, for instance, DNA may be degraded by exposing the lysate to a specific or a nonspecific endonuclease, e.g., one that preferentially acts on DNA relative to RNA. For example, the endonuclease may include a DNAse (a deoxyribonuclease) such as DNAse I, DNAse II, DNAse IV, UvrABC endonuclease, or the like. As another example, the DNA may be degraded via exposure to a restriction endonuclease. Many such nucleases are available commercially.

The lysate may be treated to separate nucleic acids. A variety of techniques can be used for such separation, including organic extraction (e.g., phenol, chloroform, and/or isoamyl alcohol), centrifugation, salting-out techniques (e.g., using potassium acetate or ammonium acetate), filtration, magnetic clearing, cesium chloride (CsCl) density gradients, solid-phase anion-exchange chromatography, binding to a solid-phase support (e.g., anion-exchange or silica), or the like. In one embodiment, for example, the lysate may be exposed to a nucleic acid separation column, e.g., containing silica. Many such columns are commercially available, and are typically used to separate DNA (not RNA) from cells. Non-nucleic acid species may be removed from the column, e.g., using suitable “wash” steps (e.g., using guanidine salts, ethanol, or the like), while nucleic acids (e.g., DNA and/or RNA) may subsequently be eluted from the column, e.g., using suitable elution buffers.

It should be noted that in some cases, there may be little DNA present, and/or the DNA that is present may be at least partially degraded, e.g., as discussed above, such that most of the nucleic acid eluted from the column is RNA.

Without wishing to be bound by any theory, it is believed that cells contain relatively low amounts of RNA compared to DNA. Accordingly, such nucleic acid columns have not typically been used to separate or isolate RNA, and are instead typically used to separate genomic DNA. Furthermore, nucleic acid columns are typically not used in conjunction with enzymes for degrading DNA, such as DNAses, as intentionally degrading the DNA would be expected to defeat the point of using a column to separate intact genomic DNA.

The RNA, once eluted, may be studied, purified, analyzed, amplified, stored, etc., using any of a variety of techniques known to those of ordinary skill in the art. In some cases, the RNA may comprise RNA from different species, e.g., human RNA and bacterial RNA. In some cases, for instance, the RNA may be sequenced and compared to known RNA sequences, thereby allowing determination of, for instance, human and non-human RNA within blood or other fluids. The non-human RNA may arise from bacteria or other pathogens, such as yeast, that may be present, e.g., within the blood or other fluids.

U.S. Provisional Patent Application Ser. No. 62/360,076, filed Jul. 8, 2016, entitled “Determination of RNA in Blood or Other Fluids,” by Weitz, et al., is incorporated herein by reference in its entirety.

The following examples are intended to illustrate certain embodiments of the present invention, but do not exemplify the full scope of the invention.

### Example 1

To overcome the problems of traditional methods which are mainly based on slow bacteria culture, this example illustrates bacterial RNA directly obtained from blood. One advantage of using bacterial RNA as a detection target is that there is no culturing of bacteria (especially since some types of bacteria are uncultivable). In addition, at early stages of infection, bacterial concentrations are sometimes very low, making isolation difficult. Detection of RNA can not only identify bacteria, but also in some cases test drug susceptibility due to the up/down regulated RNA expression by antibiotics.

However, there are relatively large amounts of RNA in blood cells, which could potentially interfere with the detection of bacterial RNA. In addition, in some cases, released enzymatic system may digest or degrade bacteria RNA. Due to these difficulties, there are no commercially available kits for the extraction of bacterial RNA from blood.

One non-limiting example experimental procedure is as follows.

Step 1. Lysis of red blood cells. Take a sample of blood, add 2-fold of Erythrocyte Lysis Buffer (e.g., Sigma-Aldrich), and incubate for 10-15 min on ice. Vortex occasionally during incubation. Centrifuge at 10,000 g for 5 min. Discard the supernatant.

Step 2. RNA protection. Add 500 microliters of PBS and 1 ml of RNAprotect Cell Reagent (Qiagen) to the pellet, and vortex for 5 seconds. Incubate at room temperature (about 25° C.) for 5 min, and centrifuge at 10,000 g for 5 min. Discard the supernatant.

Step 3. Lysis of bacteria and white blood cells enzymatically. Add 200 microliters of a buffer containing Tris-HCl, EDTA, lysozyme, and proteinase K to the pellet, pipet, and vortex to suspend the pellet. Then incubate at room temperature for 5 min. Vortex occasionally during incubation.

Step 4. Depletion of bacterial and host DNA. Add 10 microliters of DNAse I solution to the lysate, pipet to mix and incubate at room temperature for 5 min.

Step 5. Lysis of bacteria and white blood cells mechanically. Add 600 microliters of Buffer RLT (Qiagen) containing beta-mercaptoethanol to the solution, and vortex vigorously. Pipet lysate directly into a QIAshreadder spin column (Qiagen), then centrifuge at maximum speed for 2 min. Retain the lysate.

Step 6. RNA binding onto column. Add 600 microliters of 70% ethanol to the lysate and mix by pipetting. Pipet the solution to a new QIAamp spin column (Qiagen). Centrifuge for 15 seconds at 10,000 g.

Step 7. RNA washing. Transfer the QIAamp spin column into a new 2 ml tube, add 350 microliters of Buffer RW1 (Qiagen), centrifuge for 15 seconds at 10,000 g, and discard the flow-through.

Step 8. Further depletion of bacterial and host DNA. Add 10 microliters of DNAse I to 70 microliters of Buffer RDD (Qiagen), mix by gently inverting the tube, and centrifuge briefly to collect residual liquid. Add 80 microliters of DNAse working solution to the QIAamp spin column, and incubate for 15 min.

Step 9. RNA washing. Add 350 microliters of Buffer RW1 to the QIAamp spin column, and centrifuge for 15 seconds at 10,000 g. Discard the flow-through. Place the QIAamp spin column into a new 2 ml collection tube, pipet 500 microliters of Buffer RPE (Qiagen) onto the QIAamp spin column, and centrifuge for 15 seconds at 10,000 g. Pipet 500 microliters of buffer RPE onto the QIAamp spin column, and centrifuge at full speed (20,000 g) for 3 min. Place the QIAamp spin column into a new 2 ml collection tube, then centrifuge at full speed for 1 min.

Step 10. RNA elution. Transfer the QIAamp spin column into a 1.5 ml tube and pipet 30 microliters of RNase-free water onto the column membrane, then centrifuge at full speed for 1 min. The resulting solution contains RNA.

### Example 2

In this example, 0, 3, 30, and 300 klebsiella pneumoniae (a bacterium) were spiked into 500 microliters of blood. The RNA was purified as discussed above. One-step digital RT-PCR was then used to quantify Klebsiella pneumonia 16S rRNA in the sample.

As shown in FIG. 2, the Klebsiella pneumonia was successfully detected via the 16S rRNA. It should be noted that only some bacteria are cultivable, so some uncultivable bacteria cannot be detected using other techniques. However, techniques such as those described herein can be used to detect target sequences as long as the sequence of the bacteria is known.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the invention includes that number not modified by the presence of the word “about.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
adding an RNA-stabilizing reagent to a blood sample;
lysing cells within the blood sample to produce a lysate;
degrading DNA within the lysate; and
separating RNA from the lysate.

2. The method of claim 1, wherein the RNA-stabilizing reagent comprises ammonium sulfate.

3. The method of any one of claim 1 or 2, wherein the ammonium sulfate is added to produce a final concentration in the blood sample of no more than 20 g/100 mL.

4. The method of any one of claims 1-3, wherein the ammonium sulfate is added to produce a final concentration in the blood sample of no more than 64 mM.

5. The method of any one of claims 1-4, wherein lysing cells within the blood sample comprises exposing the cells within the blood sample to proteinase K.

6. The method of any one of claims 1-5, wherein lysing cells within the blood sample comprises exposing the cells within the blood sample to lysozyme.

7. The method of any one of claims 1-6, wherein degrading DNA within the lysate comprises exposing the lysate to a nonspecific endonuclease.

8. The method of any one of claims 1-7, wherein degrading DNA within the lysate comprises exposing the lysate to a DNAse.

9. The method of any one of claims 1-8, wherein degrading DNA within the lysate comprises exposing the lysate to DNAse I.

10. The method of any one of claims 1-9, wherein separating nucleic acids from the lysate comprises exposing the lysate to silica.

11. The method of any one of claims 1-10, wherein separating nucleic acids from the lysate comprises exposing the lysate to a nucleic acid separation column.

12. The method of any one of claims 1-11, wherein separating nucleic acids from the lysate comprises exposing the lysate to a guanidine salt.

13. The method of any one of claims 1-12, wherein separating nucleic acids from the lysate comprises exposing the lysate to ethanol.

14. The method of any one of claims 1-13, further comprising lysing red blood cells within the blood sample.

15. The method of claim 14, wherein lysing red blood cells within the blood sample comprises exposing the red blood cells to erythrocyte lysis buffer.

16. The method of any one of claim 14 or 15, wherein lysing red blood cells within the blood sample comprises exposing the red blood cells to ammonium chloride.

17. The method of any one of claims 14-16, wherein lysing red blood cells within the blood sample comprises exposing the red blood cells to EDTA.

18. The method of any one of claims 14-17, wherein lysing red blood cells within the blood sample comprises exposing the red blood cells to sodium bicarbonate.

19. The method of any one of claims 14-18, further comprising lysing the red blood cells prior to adding the RNA-stabilizing reagent.

20. The method of any one of claims 1-19, wherein lysing cells within the blood sample comprises mechanically lysing the cells.

21. The method of any one of claims 1-20, wherein lysing cells within the blood sample comprises exposing the blood sample to beta-mercaptoethanol.

22. The method of any one of claims 1-21, wherein lysing cells within the blood sample comprises exposing the blood sample to guanidine isothiocyanate.

23. The method of any one of claims 1-22, wherein the RNA comprises human RNA.

24. The method of any one of claims 1-23, wherein the RNA comprises bacterial RNA.

25. The method of any one of claims 1-24, further comprising determining the separated RNA.

26. The method of claim 25, comprising determining the species from which the separated RNA arises.

27. A method, comprising:
lysing red blood cells in a blood sample;
adding an RNA-stabilizing reagent to the blood sample;
lysing cells within the blood sample to produce a lysate;
degrading DNA within the lysate;
adding the lysate to a column containing silica;
removing non-nucleic acid species from the column; and
thereafter, eluting RNA from the column.

28. The method of claim 27, wherein the RNA-stabilizing reagent comprises ammonium sulfate.

29. The method of any one of claim 27 or 28, wherein the ammonium sulfate is added to produce a final concentration in the blood sample of no more than 20 g/100 mL.

30. The method of any one of claims 27-29, wherein the ammonium sulfate is added to produce a final concentration in the blood sample of no more than 64 mM.

31. The method of any one of claims 27-30, wherein lysing cells within the blood sample comprises exposing the cells within the blood sample to proteinase K.

32. The method of any one of claims 27-31, wherein lysing cells within the blood sample comprises exposing the cells within the blood sample to lysozyme.

33. The method of any one of claims 27-32, wherein degrading DNA within the lysate comprises exposing the lysate to a nonspecific endonuclease.

34. The method of any one of claims 27-33, wherein degrading DNA within the lysate comprises exposing the lysate to a DNAse.

35. The method of any one of claims 27-34, wherein degrading DNA within the lysate comprises exposing the lysate to DNAse I.

36. The method of any one of claims 27-35, comprising exposing the lysate to a guanidine salt.

37. The method of any one of claims 27-36, comprising exposing the lysate to ethanol.

38. The method of any one of claims 27-37, wherein lysing cells within the blood sample comprises exposing the cells to erythrocyte lysis buffer.

39. The method of any one of claims 27-38, wherein lysing cells within the blood sample comprises exposing the cells to ammonium chloride.

40. The method of any one of claims 27-39, wherein lysing cells within the blood sample comprises exposing the cells to EDTA.

41. The method of any one of claims 27-40, wherein lysing cells within the blood sample comprises exposing the cells to sodium bicarbonate.

42. The method of any one of claims 27-41, further comprising lysing the cells prior to adding the RNA-stabilizing reagent.

43. The method of any one of claims 27-42, wherein lysing cells within the blood sample comprises mechanically lysing the cells.

44. The method of any one of claims 27-43, wherein lysing cells within the blood sample comprises exposing the blood sample to beta-mercaptoethanol.

45. The method of any one of claims 27-44, wherein lysing cells within the blood sample comprises exposing the blood sample to guanidine isothiocyanate.

46. The method of any one of claims 27-45, wherein the RNA comprises human RNA.

47. The method of any one of claims 27-46, wherein the RNA comprises bacterial RNA.

48. The method of any one of claims 27-47, further comprising determining the separated RNA.

49. The method of claim 48, comprising determining the species from which the separated RNA arises.
